(19) United States
(12) Patent Application Publication
(10) Pub. No.: US 2023/0218395 A1
(43) Pub. Date: Jul. 13, 2023

(54) COMPRESSIBLE SHUNT IMPLANT

(71) Applicant: EDWARDS LIFESCIENCES CORPORATION, Irvine, CA (US)

(72) Inventors: Daniel James Murray, Orange, CA (US); Jean-Pierre Michel Rabbah, Tustin, CA (US)

(21) Appl. No.: 18/181,521

(22) Filed: Mar. 9, 2023

Related U.S. Application Data
(63) Continuation of application No. PCT/US2021/049740, filed on Sep. 9, 2021.
(60) Provisional application No. 62/706,874, filed on Sep. 15, 2020.

(51) Int. Cl. A61F 2/24

(52) U.S. Cl. CPC A61F 2/2427; A61F 2/2418

(57) ABSTRACT

A medical implant comprises a central flow portion having a first network of struts forming two or more cells, a first set of anchoring arms, and a second set of anchoring arms. The two or more cells are arranged linearly and extend from the first set of anchoring arms to the second set of anchoring arms.

### RELATED APPLICATION

This application claims the benefit of PCT/US2021/049740 filed on Sep. 9, 2021, which claims priority based on U.S. Provisional Patent Application Ser. No. 62/706,874, filed Sep. 15, 2020, and entitled COMPRESSIBLE SHUNT IMPLANT, the complete disclosures of which are incorporated herein by reference in their entireties.

### BACKGROUND

The present invention relates generally to the field of medical devices and procedures.

In percutaneous delivery systems for delivering certain medical implant devices to target locations at least in part through a patient's vasculature, certain anatomical and device dimensions can limit the size, shape, and/or configuration of medical implant devices delivered using such systems.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a medical implant comprising a central flow portion having a first network of struts forming two or more cells, a first set of anchoring arms, and a second set of anchoring arms. The two or more cells are arranged linearly and extend from the first set of anchoring arms to the second set of anchoring arms.

In some embodiments, each strut of the first network of struts is configured to bend in response to crimping pressure such that the two or more cells increase in length and decrease in width. Each strut of the first network of struts is configured to return to an original configuration in response to removal of the crimping pressure. In some embodiments, a diameter of the central flow portion is configured to reduce in response to the crimping pressure.

The first set of anchoring arms may have a second network of struts forming cells.

In some embodiments, the first set of anchoring arms includes a first anchoring arm and a second anchoring arm configured to anchor to a tissue wall using a pincer grasp. The first anchoring arm and the second anchoring arm may have an at least partially bent form. In some embodiments, the first anchoring arm and the second anchoring arm are configured to be at least partially straightened during delivery into a patient's body.

The central flow portion may be further configured to prevent in-growth of tissue through the central flow portion.

In some embodiments, the central flow portion is configured to expand in response to expansion of a tissue wall.

The central flow portion may be configured to fit at least partially within an opening in a tissue wall. The tissue wall may be situated between a first anatomical chamber and a second anatomical chamber. In some embodiments, the opening represents a blood flow path between the first anatomical chamber to the second anatomical chamber. The central flow portion may be further configured to maintain the blood flow path from the first anatomical chamber to the second anatomical chamber.

Some implementations of the present disclosure relate to a method comprising crimping a medical implant to reduce a profile of the medical implant. The medical implant comprises a network of struts forming two or more cells. Crimping the medical implant causes at least some struts of the network of struts to bend. The method further comprises inserting the medical implant into a catheter, delivering the catheter to a treatment location within a human body, and removing the medical implant from the catheter.

In some embodiments, medical implant further comprises a first set of anchoring arms and a second set of anchoring arms. The two or more cells may be arranged linearly from the first set of anchoring arms to the second set of anchoring arms.

Removing the medical implant from the catheter can cause expansion of the medical implant to an expanded profile. A width of the medical implant in the expanded profile may exceed a width of the catheter.

In some embodiments, the medical implant is at least partially composed of Nitinol.

Some implementations of the present disclosure relate to a medical implant comprising a continuous network of struts forming a cylindrical central flow portion allowing blood flow through an interior lumen of the central flow portion, a first set of anchoring arms extending from the central flow portion, and a second set of anchoring arms extending from the central flow portion. The central flow portion comprises a first set of two or more cells extending linearly between a first side of the first set of anchoring arms and a first side of the second set of anchoring arms.

The central flow portion may further comprise a second set of two or more cells extending linearly between a second side of the first set of anchoring arms and a second side of the second set of anchoring arms. In some embodiments, the first set of anchoring arms and the second set of anchoring arms are connected to each other only via the first set of two or more cells and the second set of two or more cells.

The first set of anchoring arms may comprise a first anchoring arm and a second anchoring arm configured to bend at least partially to establish a pincer grasp at a tissue wall.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 illustrates several access pathways for maneuvering guidewires and/or catheters in and around the heart to deploy compressible implants in accordance with some embodiments.

FIG. 2 depicts a procedure for deploying implants in accordance with some embodiments.

FIGS. 3A and 3B illustrate one example of a compressible implant in accordance with one or more embodiments.

FIGS. 4A-4C illustrate another example compressible implant in accordance with some embodiments.

FIG. 5 illustrates a delivery system for delivering one or more implants in accordance with one or more embodiments.

FIGS. 6A and 6B illustrate stages of a shape-setting process for an implant in accordance with some embodiments.

FIG. 7 (FIGS. 7-1 and 7-2) is a flow diagram illustrating a process for delivering and/or anchoring an implant to a treatment site in accordance with one or more embodiments of the present disclosure.

FIG. 8 (FIGS. 8-1 and 8-2) provides several images associated with the process of FIG. 7 to illustrate aspects of the process according to one or more implementations thereof.

### DETAILED DESCRIPTION

The headings provided herein are for convenience only and do not necessarily affect the scope or meaning of the claimed invention.

### Overview

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

The following includes a general description of human cardiac anatomy that is relevant to certain inventive features and embodiments disclosed herein and is included to provide context for certain aspects of the present disclosure. In humans and other vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and are each mounted in an annulus comprising dense fibrous rings attached either directly or indirectly to the atrial and ventricular muscle fibers. Each annulus defines a flow orifice. The four valves ensure that blood does not flow in the wrong direction during the cardiac cycle; that is, to ensure that the blood does not back flow through the valve. Blood flows from the venous system and right atrium through the tricuspid valve to the right ventricle, then from the right ventricle through the pulmonary valve to the pulmonary artery and the lungs. Oxygenated blood then flows through the mitral valve from the left atrium to the left ventricle, and finally from the left ventricle through the aortic valve to the aorta/arterial system.

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

Pulmonary hypertension (PH) is defined as a rise in mean pressure in the main pulmonary artery. PH may arise from many different causes, but, in all patients, has been shown to increase mortality rate. A deadly form of PH arises in the very small branches of the pulmonary arteries and is known as Pulmonary Arterial Hypertension (PAH). In PAH, the cells inside the small arteries multiply due to injury or disease, decreasing the area inside of the artery and thickening the arterial wall. As a result, these small pulmonary arteries narrow and stiffen, causing blood flow to become restricted and upstream pressures to rise. This increase in pressure in the main pulmonary artery is the common connection between all forms of PH regardless of underlying cause. Despite previous attempts, there is a need for an improved way to reduce elevated pressure in the left atrium, as well as other susceptible heart chambers such as the pulmonary artery.

The present disclosure provides methods and devices for delivering implants and/or similar devices to desired locations within a human body. The term “implant” is used herein according to its plain and ordinary meaning and may refer to any medical implant, frame, valve, shunt, stent, anchor, and/or similar devices for use in treating various conditions in a human body. Implants may be delivered via catheter (i.e., transcatheter) for various medical procedures and may have a generally sturdy and/or flexible structure. The term “catheter” is used herein according to its broad and ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas. In some cases, an implant may be composed of a shape-memory alloy (e.g., Nitinol) and/or may have a pre-defined shape and/or structure. The implant may be configured to be shaped and/or compressed to fit into a catheter. In some cases, an implant may have an elliptical and/or cylindrical form and/or may comprise an interweaving pattern of materials.

Conventional designs and/or related compression methods of implants (e.g., Nitinol implants) may have a variety of limitations. For example, some implants may be compressible to a given profile that may not allow the implant to fit into some catheters. The term “profile” is used herein according to its plain and ordinary meaning and may refer to one of and/or a combination of a width, surface area, diameter, radius, length, height, depth, and/or other measurement of a device and/or object. Moreover, size reduction using certain compression methods, including crimping and/or sheathing, may create stresses within the implant. When the implant is compressed beyond certain limits for the implant, the implant may be fractured and/or permanently deformed. For example, a Nitinol implant may be configured to have a pre-defined shape but may be crimped to form a smaller profile to fit into a catheter. When the implant is removed from the catheter, the implant may return to the pre-defined shape unless the compression process fractured and/or deformed the implant to an extent that prevents the implant from naturally returning to the pre-defined shape. Accordingly, the compression profile for the implant may be limited to a size at which permanent deformation of the implant is prevented. However, such a compression profile may prevent the implant from being compatible with certain catheters.

Some embodiments of the present disclosure provide implants and/or compression methods for implants with minimal stress and/or strain to the material of the implants. In some embodiments, an implant may be crimped and/or otherwise compressed to achieve a relatively small profile with respect to conventional methods and/or generally equal profile with relatively lower material strain than conventional methods.

An implant may be gradually compressed from an uncompressed form to a compressed form and/or may be compressed in multiple stages. In the uncompressed form, the implant may have a larger profile than a delivery catheter and/or an inner lumen of the delivery catheter. The implant may be compressed until the profile of the implant is approximately equal to or less than a size of the delivery catheter and/or the inner lumen of the delivery catheter. When the implant is removed from the catheter, the implant may naturally return to the uncompressed form and/or expanded profile and/or may be manually expanded and/or assisted in expansion to any form through various means. Details of these methods, implants and deployment systems will be described below.

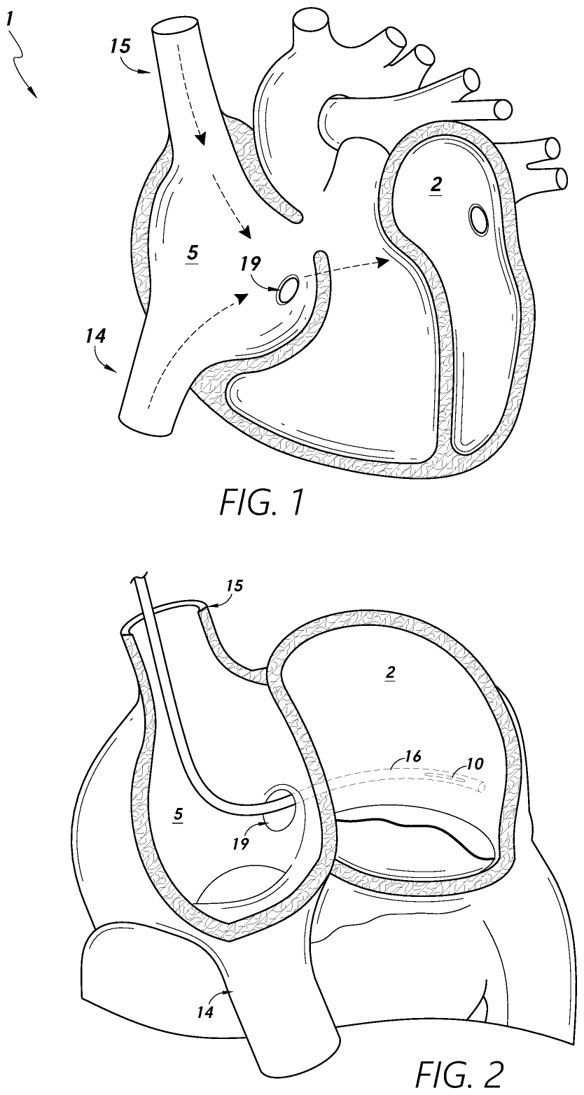

FIG. 1 illustrates several access pathways for maneuvering guidewires and catheters in and around the heart 1 to deploy compressible medical implants (e.g., frames) of the present application. For instance, access may be from above via either the subclavian vein or jugular vein into the superior vena cava (SVC) 15, right atrium (RA) 5 and from there into the coronary sinus (CS) 19. Alternatively, the access path may start in the femoral vein and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

FIG. 2 depicts an example method for deploying the medical implants 10 described herein, wherein a guidewire and/or catheter 16 is/are introduced through the subclavian or jugular vein, through the SVC 15 and into the coronary sinus 19. In some instances, a guidewire may be used to provide a path, after which an introducer sheath (not shown) may be routed along the guidewire and into the patient's vasculature, typically with the use of a dilator. FIG. 2 shows a deployment catheter 16 extending from the SVC 15 to the coronary sinus 19 of the heart 1, the deployment catheter 16 having been passed through the introducer sheath which provides a hemostatic valve to prevent blood loss.

In one embodiment, the deployment catheter 16 may be about 30 cm long, and the guidewire may be somewhat longer for ease of use. In some embodiments, the deployment catheter may function to form and prepare an opening in the wall of the left atrium 2, and a separate placement or delivery catheter will be used for delivery of an expandable implant 10. In other embodiments, the deployment catheter may be used as both the puncture preparation and implant placement catheter with full functionality. In the present application, the terms “deployment catheter” or “delivery catheter” will be used to represent a catheter or introducer with one or both of these functions.

Since the coronary sinus 19 is largely contiguous around the left atrium 2, there are a variety of possible acceptable placements for implants 10. The site selected for placement of the stent, may be made in an area where the tissue of the particular patient is less thick or less dense, as determined beforehand by non-invasive diagnostic means, such as a CT scan or radiographic technique, such as fluoroscopy or intravascular coronary echo (IVUS).

Some methods to reduce LAP involve utilizing an implant 10 between the left atrium 2 and the right atrium 5, through the interatrial septum therebetween. This is a convenient approach, as the two structures are adjacent and transseptal access is common practice. However, there may be a possibility of emboli travelling from the right side of the heart to the left, which presents a stroke risk. This event should only happen if the right atrium pressures go above left atrium pressures; primarily during discrete events like coughing, sneezing, Valsalva maneuver, or bowel movements. The anatomical position of the septum would naturally allow emboli to travel freely between the atria if an implant 10 was present and the pressure gradient flipped. This can be mitigated by a valve or filter element in the implant 10, but there may still be risk that emboli will cross over.

Implanting to the coronary sinus 19 offers some distinct advantages, primarily that the coronary sinus 19 is much less likely to have emboli present for several reasons. First, the blood draining from the coronary vasculature into the right atrium 5 has just passed through capillaries, so it is essentially filtered blood. Second, the ostium of the coronary sinus 19 in the right atrium 5 is often partially covered by a pseudo-valve called the Thebesian Valve. The Thebesian Valve is not always present, but some studies show it is present in >60% of hearts and it would act as a natural “guard dog” to the coronary sinus to prevent emboli from entering in the event of a spike in right atrium pressure. Third, pressure gradient between the coronary sinus 19 and the right atrium 5 into which it drains is very low, meaning that emboli in the right atrium 5 is likely to remain there. Fourth, in the event that emboli do enter the coronary sinus 19, there will be a much greater gradient between the right atrium 5 and the coronary vasculature than between the right atrium 5 and the left atrium 2. Most likely emboli would travel further down the coronary vasculature until right atrium pressure returned to normal and then the emboli would return directly to the right atrium 5.

Some additional advantages to locating the implant 10 between the left atrium 2 and the coronary sinus 19 is that this anatomy is less mobile than the septum (it is more stable), it thus preserves the septum for later transseptal access for alternate therapies, and it could potentially have other therapeutic benefits. By diverting left atrial blood into the coronary sinus 19, sinus pressures may increase by a small amount. This would cause blood in the coronary vasculature to travel more slowly through the heart, increasing perfusion and oxygen transfer, which would be more efficient and also could help a dying heart muscle to recover. The preservation of transseptal access also is a very significant advantage because HF patients often have a number of other comorbidities like Atrial Fibrillation (AF) and Mitral Regurgitation (MR) and several of the therapies for treating these conditions require a transseptal approach.

An implant 10 may also be positioned within chambers and/or vessels and/or between other cardiac chambers, such as between the pulmonary artery and right atrium 5. The implant 10 may be desirably implanted within the wall of the pulmonary artery using the deployment tools described herein, with the catheters approaching from above and passing through the pulmonary artery. As explained above, pulmonary hypertension (PH) is defined as a rise in mean pressure in the main pulmonary artery. Blood flows through the implant 10 from the pulmonary artery into the right atrium 5 if the pressure differential causes flow in that direction, which attenuates pressure and reduces damage to the pulmonary artery. The purpose is to attenuate pressure spikes in the pulmonary artery. The implant 10 may also extend from the pulmonary artery to other heart chambers (e.g., left atrium 2) and/or blood vessels. In some embodiments, the implant 10 may further contain a one-way valve for preventing backflow, or a check valve for allowing blood to pass only above a designated pressure.

Some implants 10 described herein may be at least partially compressible and/or expandable. Moreover, in some embodiments, an implant 10 may have various features and/or may be used in combination with devices having various barriers for preventing, inhibiting, and/or containing tissue growth. The implant 10 may be configured to at least partially prevent, inhibit, reduce, contain, and/or otherwise alter tissue growth and/or in-growth of tissue at and/or around the implant 10 and/or within an opening in a tissue wall. Implants 10 described herein may have various features to simplify and/or improve delivery procedures for surgeons. For example, an implant 10 may be at least partially flexible, compressible, and/or elastic to allow the implant 10 to be shaped and/or molded as necessary/desired to fit into delivery catheters having various sizes and/or shapes.

Moreover, an implant 10 may be configured to various openings created in tissue walls having various sizes and/or shapes. A tissue wall may be situated between a first anatomical chamber (e.g., the coronary sinus) and a second anatomical chamber (e.g., the left atrium). In some embodiments, an opening may be created through the tissue wall and/or the implant 10 (e.g., a central flow portion and/or central flow portion of the implant 10) may be configured to fit at least partially within the opening. The opening may represent a blood flow path between the first anatomical chamber and the second anatomical chamber. In some embodiments, the implant 10 may be configured to maintain the opening and/or the blood flow path from the first anatomical chamber to the second anatomical chamber.

### Implant Devices

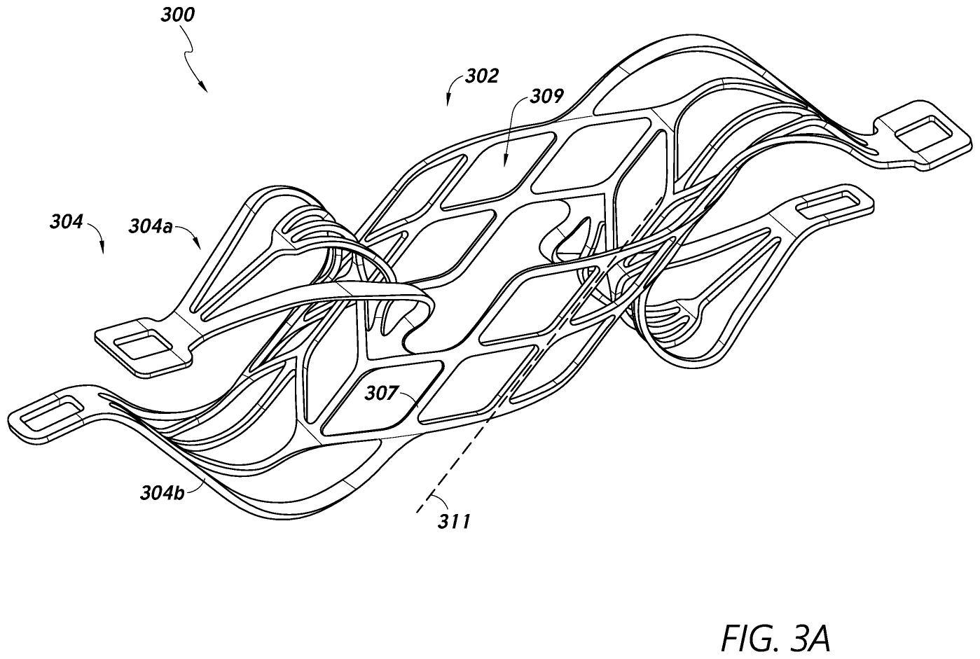

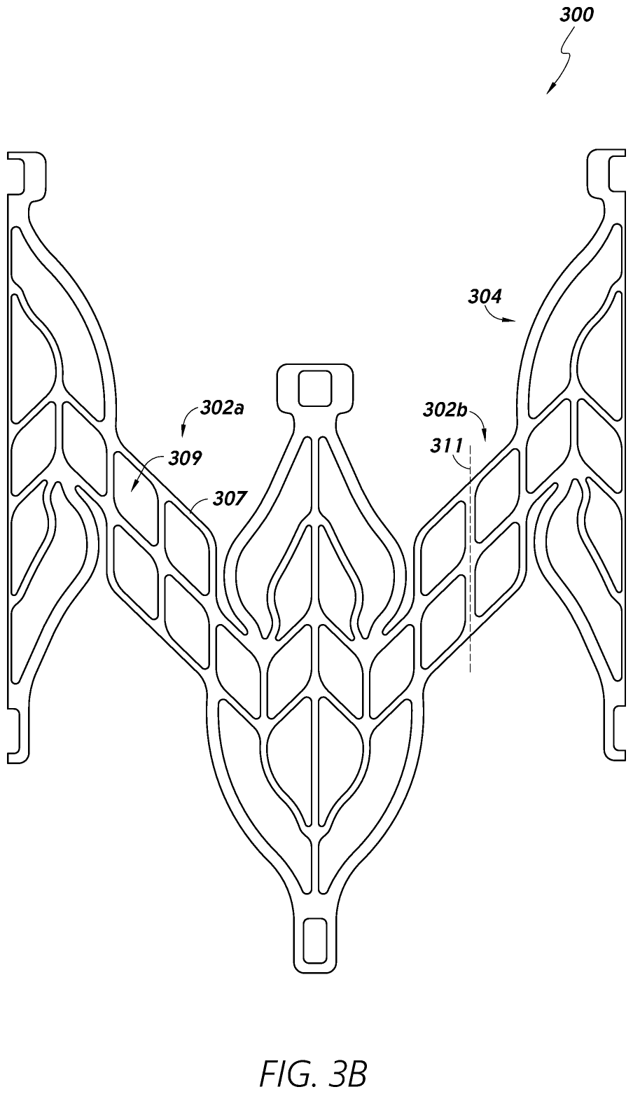

FIGS. 3A and 3B illustrate one example of a compressible implant 300. The implant 300 may comprise any of a variety of features and/or components configured to treat various medical conditions. For example, the implant 300 may be configured to maintain an opening in a tissue wall and/or allow blood flow through the tissue wall. In some embodiments, the implant 300 may comprise a central flow portion 302 which may be configured to be situated at least partially within an opening in a tissue wall. The central flow portion 302 may represent a central flow portion configured to create and/or maintain an opening between two anatomical chambers. In some embodiments, the implant 300 may comprise multiple separate components which may be attached, connected, and/or otherwise joined to form a single device. For example, the central flow portion 302 may comprise multiple components to form a generally tubular shape which may approximate a shape of an opening in a tissue wall.

In some embodiments, the implant 300 may be configured to be movable between an expanded configuration and a collapsed configuration to facilitate passage through a lumen of a catheter. For example, the central flow portion 302 may be configured to be crimped and/or otherwise compacted to fit within the lumen of the catheter. Crimping may involve a reduction in diameter of the central flow portion 302 and/or an increase in length of the implant 300. When the implant 300 is crimped, at least a portion of the implant 300 may create resistive force in response to crimping pressure. For example, crimping force may be applied along a resistance line 311 including multiple struts 607 of the implant 300.

The central flow portion 302 may be configured to expand to a pre-defined shape (e.g., the shape and/or size shown in FIG. 3A) and/or size during and/or after removal from a deliver device (e.g., a catheter). The implant 300 may further comprise one or more anchoring arms 304, which may include flanges, arms, anchors, and/or other devices. In some embodiments, the one or more anchoring arms 304 may be configured to extend generally perpendicularly (i.e., forming a “T” shape) from the central flow portion. The one or more anchoring arms 304 may have a generally flat, curved, and/or wavy form. In some embodiments, the one or more anchoring arms 304 may be configured to at least partially collapse and/or compress to facilitate passage through the lumen of the catheter and/or may be configured to expand during and/or after delivery within the body to contact and/or attach to a tissue wall. Expansion of the implant 300 may be initiated, for example, by retraction of an outer sheath of the catheter relative to an inner support sheath. The implant 300 may be collapsed (e.g., crimped) into a generally tubular configuration. In some embodiments, the anchoring arms 304 may be configured to spring open when the restraining outer sheath retracts. The anchoring arms 304 may expand generally in opposite directions in a common plane to form a T-shape (see FIG. 3B), as opposed to expanding in a circular fashion. Radiopaque markers on the anchoring arms 304 and/or central flow portion 302 may be provided to facilitate positioning immediately within the body.

A pair of anchoring arms 304 (e.g., a first anchoring arm 304a and a second anchoring arm 304b) may be configured to form a clamping (i.e., pinching) pair of anchoring arms 304. The pairs of anchoring arms 304 may be configured to apply a compressive force to a tissue wall to hold the implant 300 in place. The amount of compressive force may be relatively small to avoid damage to the tissue wall while sufficient to hold the implant 300 in place. For example, gaps separating the pairs of anchoring arms may be calibrated to avoid excessive clamping and/or necrosis of the tissue. The anchoring arms 304 may be configured to secure the implant 300 on generally opposite sides of the tissue wall and/or on generally opposite sides of an opening in the tissue wall. The central flow portion 302 may be configured to be aligned generally perpendicularly to the tissue wall so as to maintain an open flow path between the chambers on either side of the tissue wall.

Components of the implant 300 may be configured to naturally self-expand due to inherent springiness and/or flexibility of the components. For example, various components (e.g., the central flow portion 302 and/or anchoring arms 304) may be at least partially composed of an elastic material such as Nitinol. In some embodiments, the central flow portion 302 may be fabricated by laser cutting a Nitinol tube.

As shown in FIGS. 3A and 3B, the central flow portion 302 may be composed of generally thin struts 307 in a generally parallelogram arrangement that may form an array of parallelogram-shaped cells 309 or openings.

The central flow portion 302 may be configured to form a generally tubular or other shape to approximate a shape of the opening. In some embodiments, the opening may be widened in all directions approximately evenly from a puncture point to form an approximately circular opening having a certain diameter. Accordingly, the central flow portion 302, including the struts 307, may be configured to hold an at least partially rounded and/or circular form around/about the opening along a longitudinal axis (i.e., into the opening).

In some embodiments, the expandable implant 300 may be in a compacted and/or otherwise expandable form at delivery. For example, at delivery, the central flow portion 302 and/or anchoring arms 304 may be folded, bent, and/or otherwise compacted to have a minimal profile to facilitate passage through a delivery catheter. After delivery, the central flow portion 302 and/or anchoring arms 304 may be configured to unfold, unwrap, and/or otherwise expand (e.g., to form the design shown in FIG. 3A). In some embodiments, at least a portion of the central flow portion 302 and/or anchoring arms 304 may be composed of Nitinol and/or a similar material having shape-memory characteristics such that the implant 300 may naturally assume a pre-determined form after removal from the delivery catheter.

FIG. 3B illustrates another view of the example implant 300. For illustrative purposes, the implant 300 is showing lying flat with a pair of anchoring arms 304 bisected. The implant 300 may not necessarily be configured to be cut and/or formed in this way but is presented in a flat form to better illustrate the various components of the implant 300. The implant 300 may include a network of struts 307 forming one or more cells 309 and/or a central flow portion 302 configured to surround a flow path through the implant 300. A strut 307 may have a generally thin wire-like form. The central flow portion/central flow portion 302 may be configured to define a flow path between two anatomical chambers (e.g., the coronary sinus and the left atrium). The central flow portion 302 may be configured to be placed within an opening in a tissue wall between the two anatomical chambers. The central flow portion 302 may be configured to form a generally tubular form (e.g., the form shown in FIG. 3A), in which the central flow portion 302 (i.e., central flow portion) may form a complete ellipse and/or cylinder about a flow path through a tissue wall.

The implant 300 may have any of a variety of features and/or components configured to maintain an opening in a tissue wall and/or allow blood flow through the tissue wall. In some embodiments, the implant 300 may comprise a central flow portion 302 which may be configured to be situated at least partially within the opening in the tissue wall. In some embodiments, the implant 300 may comprise multiple separate components which may be attached, connected, and/or otherwise joined to form a single device. For example, the central flow portion 302 may comprise multiple components to form a generally tubular shape which may approximate a shape of the opening in the tissue wall. For example, the opening may have a generally elliptical (e.g., circular) form (see, e.g., FIG. 3A) and the central flow portion 302 may be configured to form a generally cylindrical and/or tubular form to fit within and/or press against an inner surface of the tissue wall at the opening.

However, the central flow portion 302, including the struts 307 and/or cells 309, may have any shape, size, and/or orientation. Rather than a generally parallelogram shape, the cells 309 may have a generally elliptical, triangular, hexagonal, or other shape. Moreover, the central flow portion 302 may not comprise any cells 309. In some embodiments, the shape of the struts 307, cells 309, and/or the central flow portion 302 generally may facilitate a collapsibility and/or expandability of the central flow portion 302 for passage through a lumen of a catheter.

Any of the one or more anchoring arms 304 may comprise one or more anchoring mechanisms, which may be situated, for example, at an end portion of the anchoring arm. Suitable anchoring mechanism may include any devices configured to penetrate and/or otherwise securely contact the tissue wall. For example, an anchoring mechanism may comprise one or more of a barb, a hook, a nail, and a screw. When the implant 300 is placed at the tissue wall, the anchoring mechanisms may be configured to interact with the tissue wall to securely hold the implant 300 in place.

In some embodiments, the implant 300 may be configured to be movable between an expanded configuration and a collapsed (e.g., generally tubular) configuration to facilitate passage through a lumen of a catheter. For example, the central flow portion 302 may be configured to be bent, twisted, or otherwise compacted to fit within the lumen of the catheter. The central flow portion 302 may be configured to expand to a pre-defined shape and/or size during and/or after delivery within the body. The implant 300 may further comprise one or more anchoring arms 304, which may include flanges, arms, anchors, and/or other devices. The one or more anchoring arms 304 may be configured to at least partially collapse to facilitate passage through the lumen of the catheter and may be configured to expand during and/or after delivery within the body to contact and/or attach to the tissue wall. Expansion of the implant 300 may be initiated, for example, by retraction of an outer sheath of the catheter relative to an inner sheath. The implant 300 may be collapsed (e.g., crimped) into a generally tubular configuration between the two sheaths with the anchoring arms 304 straightened, and the anchoring arms 304 may be configured to spring open when the restraining outer sheath retracts. The anchoring arms 304 may expand generally in opposite direction in a common plane to form a T-shape, as opposed to expanding in a circular fashion. Radiopaque markers on the anchoring arms 304 may be provided to facilitate positioning immediately within the left atrium.

A pair of anchoring arms 304 (e.g., a first anchoring arm 304a and a second anchoring arm 304b) may form a clamping (i.e., pinching) pair of anchoring arms. The pairs of anchoring arms 304 may be configured to apply a compressive force to the tissue wall to hold the implant 300 in place. The amount of compressive force may be relatively small to avoid damage to the tissue wall while sufficient to hold the implant 300 in place. For example, gaps separating the pairs of anchoring arms may be calibrated to avoid excessive clamping and/or necrosis of the tissue. The anchoring arms 304 may be configured to secure the implant 300 on generally opposite sides of the tissue wall (e.g., the first anchoring arm 304a on a first side of the tissue wall and the second anchoring arm 304b on a second side of the tissue wall) and/or on generally opposite sides of the opening in the tissue wall. The central flow portion 302 may be configured to be aligned generally perpendicular to the tissue wall so as to maintain an open flow path between the chambers on either side of the tissue wall (e.g., the coronary sinus and the left atrium).

Components of the implant 300 may be configured to naturally self- expand due to inherent springiness and/or flexibility of the components. For example, various components (e.g., the central flow portion 302 and/or anchoring arms 304) may be composed of an elastic material such as Nitinol. In some embodiments, the central flow portion 302 may be fabricated by laser cutting a Nitinol tube. The central flow portion 302 may have a wall thickness of between about 0.1-0.3 mm.

The implant 300 may be configured to be compressed (e.g., crimped), which may involve reducing a diameter of the central flow portion 302. At least some components of the implant 300 may be configured to at least partially bend and/or otherwise conform in response to compression force. For example, compression force may be applied at least along

As shown in FIGS. 3A and 3B, the central flow portion 302 may be composed of generally thin struts 307 in a generally parallelogram arrangement that may form an array of parallelogram-shaped cells 309 or openings. However, the central flow portion 302, including the struts 307 and/or cells 309, may have any shape, size, and/or orientation. For example, the struts 307 may have a generally thicker design than shown in FIGS. 3A and 3B to minimize the size of the cells 309, thereby further preventing in-growth of tissue through the central flow portion 302. Rather than a generally parallelogram shape, the cells 309 may have a generally elliptical, triangular, hexagonal, or other shape. Moreover, the central flow portion 302 may not comprise any cells 309. In some embodiments, the shape of the struts 307, cells 309, and/or the central flow portion 302 generally may facilitate a collapsibility and/or expandability of the central flow portion 302 for passage through a lumen of a catheter.

The flow portion 302 may be configured to form a generally cylindrical or other shape to approximate a shape of the opening. In some embodiments, the opening may be widened in all directions approximately evenly from a puncture point to form an approximately circular opening having a certain diameter. Accordingly, the flow portion 302, including the struts 307, may have an at least partially rounded and/or circular form around/about the opening along a longitudinal axis (i.e., into the opening).

In some embodiments, the expandable implant 300 may be in a compacted and/or otherwise expandable form at delivery. For example, at delivery, the central flow portion 302 and/or anchoring arms may be folded, bent, and/or otherwise compacted to have a minimal profile to facilitate passage through a delivery catheter. After delivery, the central flow portion 302 and/or anchoring arms 304 may be configured to unfold, unwrap, and/or otherwise expand (e.g., to form the design shown in FIG. 3A). In some embodiments, at least a portion of the central flow portion 302 and/or anchoring arms 304 may be composed of Nitinol and/or a similar material having shape-memory characteristics such that the implant 300 may be configured to naturally assume a pre-determined form after removal from the delivery catheter.

Moreover, the central flow portion 302 and/or anchoring arms 304 may be configured to expand in response to growth and/or expansion of the tissue wall. For example, as the tissue wall expands (i.e., thickens), the first anchoring arm 304a and the second anchoring arm 304b may be configured to separate further from each other to some extent to accommodate the growth of the tissue wall. In some embodiments, the central flow portion 302 and/or anchoring arms 304 may be configured to stretch in response to expansion of the tissue wall. For example, the central flow portion 302 and/or anchoring arms 304 may be at least partially composed of a flexible and/or elastic material that may allow for some amount of stretching. As the tissue wall expands, the implant 300 may be configured to stretch to accommodate the expansion of the tissue wall.

The central flow portion 302 may comprise a first portion 302a and a second portion 302b. Each of the first portion 302a and the second portion 302b may be configured to extend between the same two sets of anchoring arms 304. The first portion 302a and the second portion 302b may be configured to form opposing sides of an interior lumen through the implant 300. The two sets of anchoring arms 304 may complete the sides of the interior lumen such that the interior lumen (e.g., flow passage) through the implant 300 has a generally cylindrical and/or elliptical shape.

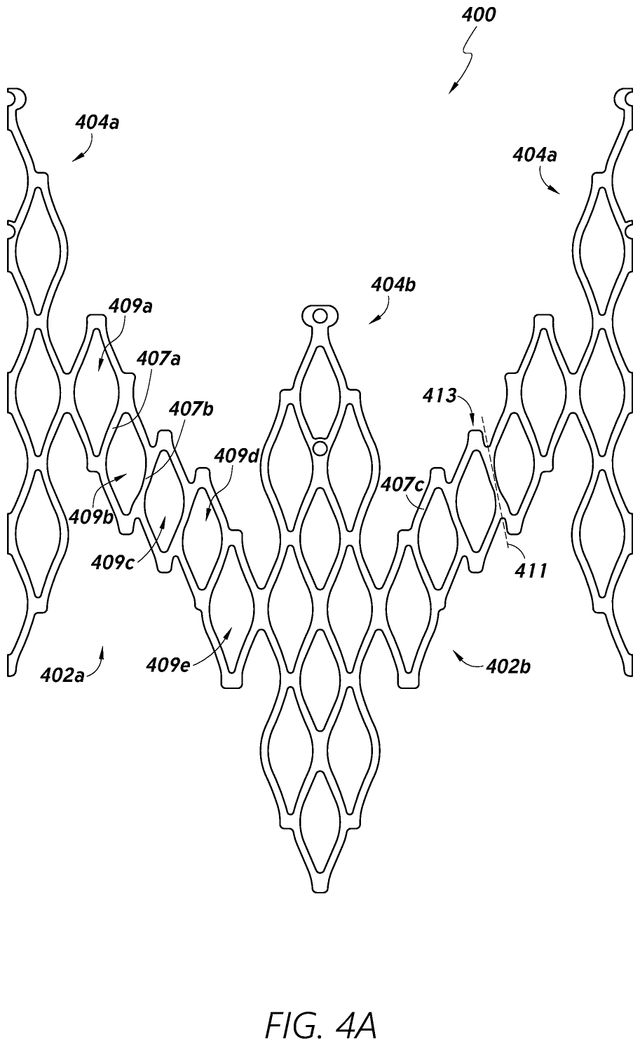

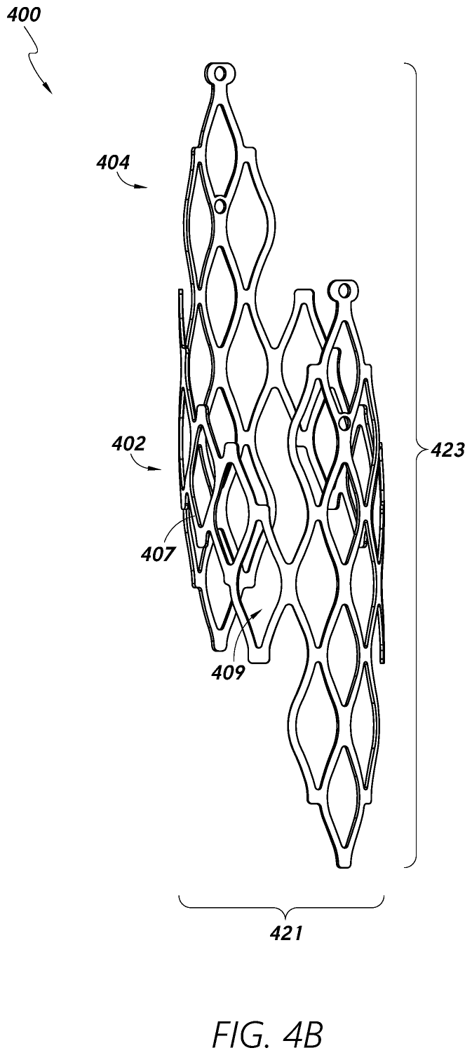

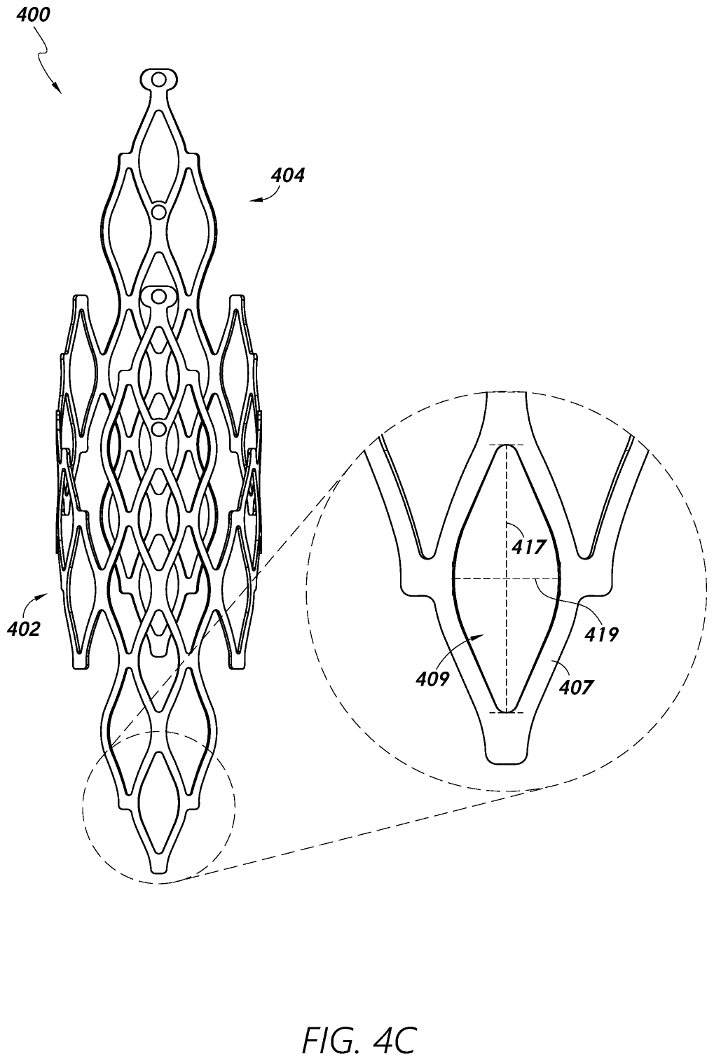

FIGS. 4A-4C illustrate another example compressible implant 400. For illustrative purposes, FIG. 4A shows the implant 400 lying flat with a pair of anchoring arms 404 bisected. The implant 400 may not necessarily be configured to be cut and/or formed in this way but is presented in a flat form to better illustrate the various components of the implant 400. The implant 400 may include a network of struts 407 forming one or more cells 409 and/or a central flow portion 402 configured to surround a flow path through the implant 400. The term “strut” is used herein according to its plain and ordinary meaning and may refer to any generally thin length of material configured to form a structure of a medical implant. The network of struts 407 may have a mesh-like form in which various struts 407 interconnect around a pattern of cells 409, which can include openings and/or gaps in the network of struts 407.

The implant 400 depicted in FIGS. 4A-4C may be configured to be used interchangeably and/or to perform the same function as the implant 300 described above with respect to FIGS. 3A and 3B. For example, the implant 400 depicted in FIGS. 4A-4C and the implant 300 depicted in FIGS. 3A and 3B may have similar and/or identical geometries. The above description above related to FIGS. 3A and 3B may be applied to the implant 400 shown in FIGS. 4A-4C. Thus, the implant 400 may comprise any of a variety of features and/or components configured to treat various medical conditions. For example, the implant 400 may be configured to maintain an opening in a tissue wall and/or allow blood flow through the tissue wall. In some embodiments, the implant 400 may comprise a central flow portion 402 (or “means for maintaining a flow path”) which may be configured to be situated at least partially within an opening in a tissue wall. The central flow portion 402 may represent a central flow portion configured to create and/or maintain an opening between two anatomical chambers. In some embodiments, the implant 400 may comprise multiple separate components which may be attached, connected, and/or otherwise joined to form a single device. For example, the central flow portion 402 may comprise multiple components to form a generally tubular shape which may approximate a shape of an opening in a tissue wall.

Patients receiving the various medical implants 400 described herein may have varying anatomies. Thus, it may be advantageous for a medical implant 400 to be as small as possible and/or to be able to be compressed to a smallest possible profile for delivery into the patient's body. For example, a larger implant 400 may not be able to be passed through relatively small anatomical structures while a relatively small implant 400 may be able to fit through more variously-sized anatomical structures.

As shown in FIG. 4A, the central flow portion 402 of the implant 400 may include two series (402a and 402b) of generally linearly connected cells 409 extending between a first set of anchoring arms 404a (or “means for anchoring”) and a second set of anchoring arms 404b. Each cell 409 may form a four-sided shape. The cells 409 forming the central flow portion 402 may be adjoined to a maximum of two other cells 409. For example, in a first portion 402a of the central flow portion 402, a first cell 409a may be separated from a second cell 409b by a first strut 407a. Similarly, the second cell 409b may be separated from a third cell 409c by a second strut 407b. Additional cells, including a fourth cell 409d and/or fifth cell 409e, may also be included in the first portion 402a and may also be separated by a single strut 407. Some struts 407 around cells 409 of the central flow portion 402, including a third strut 407c, may not be situated between cells 409.

One or more cells 409 may be generally aligned in series between the first anchoring arms 404a and the second anchoring arms 404b. The alignment of the various cells 409 and/or struts 407 may be configured such that, when crimping force is applied to the implant 400, a line of tension 411 may be created between two cells 409. The line of tension 411 may present minimal resistance to crimping and/or other compression of the implant 400, as the line of tension 411 may extend only along a connective strut 407 between two cells 409.

The network of struts 407 may form a pattern in which multiple cells 409 are at least partially aligned. The pattern of struts 407 may include one or more edges 413 extending outwardly from the cells 409 at various points. As shown in FIG. 4A, the various cells 409 in a series of cells may not be perfectly aligned and/or may be offset at least partially from neighboring cells 409. For example, a straight line passing through a central point of a first cell 409a and a central point of a second cell 409b may pass through a third cell 409c but not at a central point of the third cell 409c. The various cells 409 may be at least partially offset from each other as needed to establish a desired orientation between the first set of anchoring arms 404a and the second set of anchoring arms 404b. The orientation between the first set of anchoring arms 404a and the second set of anchoring arms 404b may be determined based on the size/shape of the tissue wall and/or opening in the tissue where the implant 400 it to be delivered to.

The network of struts 407 may be a continuous network as shown in FIG. 4B. The central flow portion 402 may have a continuous and/or generally cylindrical form about an interior lumen to allow blood flow through the interior lumen. The first set of anchoring arms 404a and/or the second set of anchoring arms 404b may be configured to extend from the central flow portion. The central flow portion 402 can comprise two sets of two or more cells extending linearly between the first set of anchoring arms 404a and the second set of anchoring arms 404b.

A first set of two or more cells (e.g., cells 409a-409e and/or other cells 409 of the first portion 402a) may be configured to extend linearly between a first side of the first set of anchoring arms 404a and a first side of the second set of anchoring arms 404b. The second set of cells (e.g., the cells 409 of the second portion 402b) may be configured to extend linearly between a second side of the first set of anchoring arms 404a and a second side of the second set of anchoring arms 404b. The first set of anchoring arms 404a and the second set of anchoring arms 404b may be connected to each other only via the first set of two or more cells 409 and the second set of two or more cells 409. The first set of anchoring arms 404a can comprise a first anchoring arm and a second anchoring arm configured to bend at least partially to establish a pincer grasp at a tissue wall.

The cells 409 forming the central flow portion 402 may be configured to be arranged in a single-file manner such that bending the central flow portion 402 requires bending only singular strut 407 portions between two cells 409 (e.g., along the tension line 411). In the example shown in FIG. 4A, the first portion 402a and the second portion 402b may include five cells 409 each arranged in a generally linear manner. The cells 409 of the first portion 402a and/or second portion 402b may extend diagonally, with respect to the sets of anchoring arms 404a, 404b between the first set of anchoring arms 404a and the second set of anchoring arms 404b. The various struts 407 may extend continuously from the central flow portion 402 to the anchoring arms 404. In some embodiments, a series of cells 409 (e.g., four cells) may extend horizontally and/or may bisect a first anchoring arm and a second anchoring arm of the first set of anchoring arms 404a and/or of the second set of anchoring arms 404b.

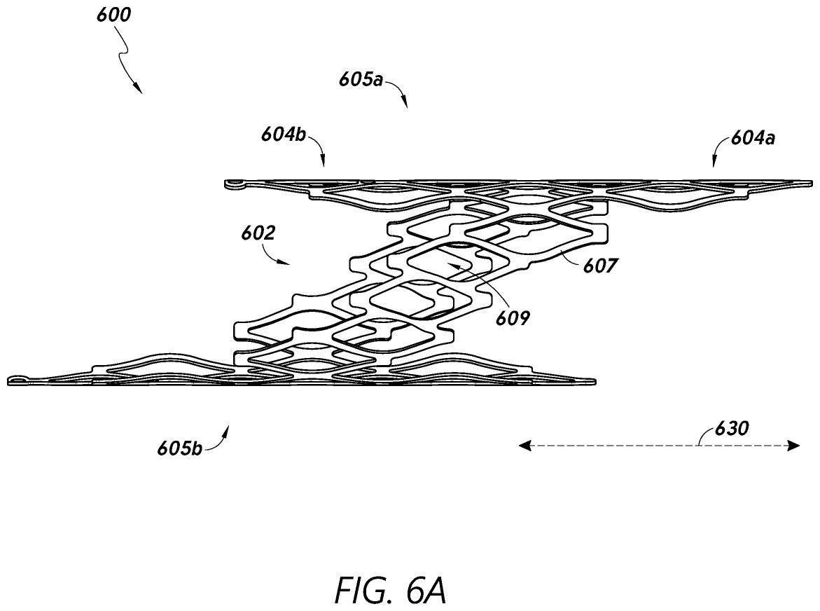

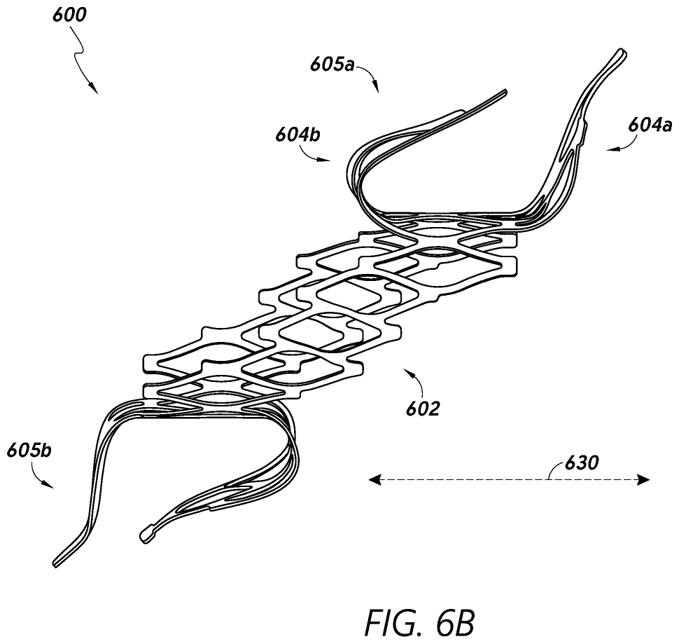

FIG. 4B illustrates the implant 400 as a continuous device having a generally tubular form about an interior lumen of the implant 400. The central flow portion 402 may be configured to form an outer wall including a network of struts 407 forming one or more cells 409 around the interior lumen. The anchoring arms 404 are shown in a flattened form, in which the anchoring arms 404 are not bent to attach to a tissue wall. In some embodiments, the implant may be delivered in the flattened form shown in FIG. 4B or may be delivered with the anchoring arms at least partially bent (see, e.g., FIG. 6B).

The implant 400 may have a width 421 (i.e., diameter) measuring across the central flow portion 402 of the implant 400. The implant 400 may also be characterized by a length 423 measuring from one set of anchoring arms 404 to another set of anchoring arms 404. When the implant 400 is crimped, the width 421 of the implant 400 may be decreased and/or the length 423 of the implant 400 may be increased. Similarly, when the implant 400 is expanded (e.g., upon exiting a deliver device such as a catheter), the width 421 of the implant 400 may be increased and/or the length 423 of the implant 400 may be decreased.

As shown in FIG. 4B, the struts of the network of struts 407 forming the central flow portion 402 and/or the anchoring arms 404 may be at least partially bent to form the cylindrical shape of the implant 400. In response to crimping pressure, the amount of bend of the struts may be configured to increase to increase the length 423 and/or to decrease the width 421 of the implant 400.

The shape and/or form of the implant 400 shown in FIG. 4B may be an original and/or natural shape and/or form of the implant 400. For example, the implant 400 may have a first width/diameter 421 prior to delivering into a patient's body. In some cases, the width/diameter 421 of the implant 400 in the original and/or natural configuration may exceed a diameter of a delivery device (e.g., a catheter). During delivery, the implant 400 may be crimped and/or various struts 407 of the implant may be at least partially bent, causing the width/diameter 421 to decrease and/or causing the length 423 to increase. When the implant 400 reaches a target/treatment location within the patient's body, the implant 400 may be removed from a delivery device (e.g., a catheter). In some embodiments, removal from the delivery device may cause the implant 400 to naturally assume the original/natural form shown in FIG. 4B, with the original width/diameter 421 and/or length 423. The implant 400 may additionally or alternatively be assisted to expand to the original/natural form using various instruments.

In some embodiments, the anchoring arms 404 of the implant 400 may be configured to assume a straight form and/or may be configured to be straightened as shown in FIG. 4B before, during, and/or after delivery into a patient's body. In a straightened configuration, each anchoring arm 404 of a pair of anchoring arms 404 may be situated as a generally continuous line along the implant 400. By straightening the anchoring arms 404, the profile of the implant 400 may be reduced to more effectively fit the implant 400 into a delivery device (e.g., a catheter). For example, a pair of anchoring arms 404 may be shape-set to form a pincer grasp for anchoring to a tissue wall. With the anchoring arms 404 forming the pincer grasp, the width 421 of the implant 400 may be increased. However, straightening the anchoring arms 404 may greatly reduce the width 421 of the implant 400.

FIG. 4C illustrates another view of the implant 400 in its continuous form. The implant may comprise a network of struts 407 forming any number of cells 409. In some embodiments, both the central flow portion 402 and the anchoring arms 404 of the implant 400 may include struts 407 forming cells 409. The cells 409 formed in the central flow portion 402 may be similar or identical to the cells 409 formed in the anchoring arms 404. However, in some embodiments, the anchoring arms 404 may not form cells 409 and/or may form differently sized and/or shaped cells 409 than the central flow portion 402.

Each cell 409 may form any shape, including the rounded rhombus/elliptical form shown in FIGS. 4A-4C. Each cell 409 may have a cell length 417 and a cell width 419. The cell length 417 may be greater than the cell width 419. The network of struts 407 forming the central flow portion 402 and/or the anchoring arms 404 may be configured to be at least partially flexible such that, in response to crimping force, the cell length 417 may be configured to increase and/or the cell width 419 may be configured to decrease. Similarly, in response to removal of crimping force (e.g., removal from a catheter or other delivery device), the cell length 417 may be configured to decrease and/or the cell width 419 may be configured to increase.

The cells 409 forming the central flow portion 402 may be arranged linearly. For example, it may be possible to draw a line through each of the cells 409 (e.g., 409a-409e) forming a first portion 402a of the central flow portion 402 and/or to draw a line through each of the cells 409 forming a second portion 402b of the central flow portion 402. In some embodiments, the first portion 402a and/or the second portion 402b may be configured to extend from the first set of anchoring arms 404a to the second set of anchoring arms 404b. That is, a line connecting the first set of anchoring arms 404a to the second set of anchoring arms 404b may pass through all cells 409 within the first portion 402a or second portion 402b extending from the first set of anchoring arms 404a to the second set of anchoring arms 404b.

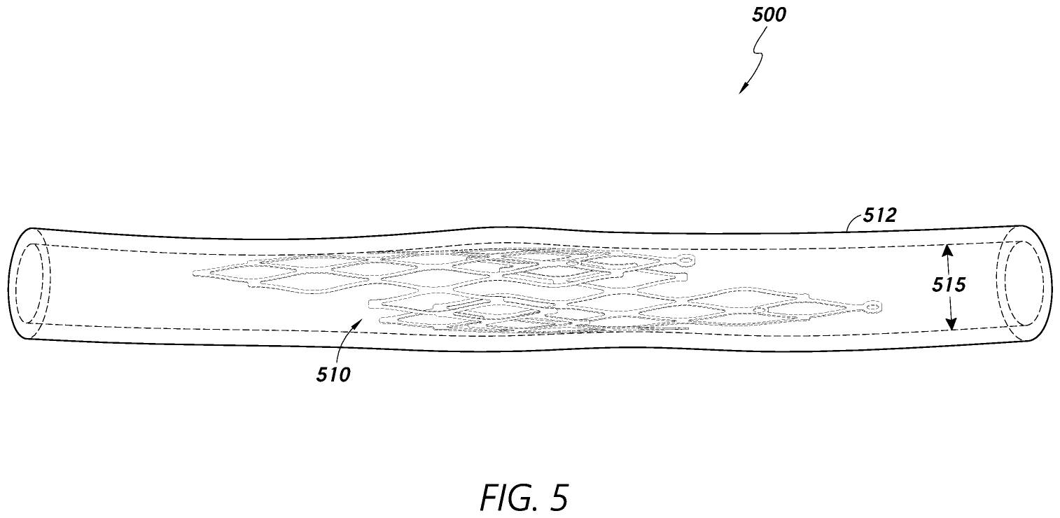

FIG. 5 illustrate a delivery system 500 for delivering one or more implants 510 in accordance with one or more embodiments. One or more implants 510 may be situated within an inner lumen of a catheter 512. At delivery, the implant 510 may be in a compressed form and/or may have a reduced and/or minimal profile. For example, an inner wall of the catheter 512 may press against the implant 510 to prevent the implant 510 from expanding to an expanded profile. In some embodiments, the implant 510 may be configured to expand when the implant 510 is removed from the catheter 512. For example, the implant 510 may be at least partially composed of a shape-memory alloy (e.g., Nitinol) and/or may be configured to naturally assume a pre-defined shape. In some embodiments, the implant 510 may be configured to be manually shaped and/or otherwise assisted in expanding after exiting the catheter 512.

The catheter 512 may have an inner diameter 515 that is less than a diameter/width of the implant 510 in an expanded form (see, e.g., FIG. 4B). However, the implant 510 may be configured to compress in response to crimping force. The structure of the central flow portion of the implant 510 may facilitate compression of the implant 510.

FIGS. 6A and 6B illustrate stages of a shape-setting process for an implant 600. In some embodiments, the implant 600 may be formed through use of a laser-cutting process. One or more anchoring arms 604 of the implant 600 may be formed in the generally straight configuration shown in FIG. 6A or in the at least partially bent/curved configuration shown in FIG. 6B.

The implant 600 may be at least partially composed of a network of struts 607 forming one or more cells 609. In some embodiments, at least a portion of the struts 607 may be composed of a shape-memory alloy and/or other flexible material. For example, the implant 600 may be at least partially composed of Nitinol.

In some embodiments, the implant 600 may be configured to form the orientation shown in FIG. 6A, in which a first set 605a of anchoring arms 604 (including a first anchoring arm 604a and a second anchoring arm 604b) and/or a second set 605b of anchoring arms 604 may be generally flat and/or may form a continuous approximately straight line along the implant 600. The implant may be shape-set in the form shown in FIG. 6B, in which the first anchoring arm 604a and/or the second anchoring arm 604b may be at least partially bent and/or curved towards each other.

During a delivery process, the implant may be pressed into the form shown in FIG. 6A to facilitate easier insertion into a delivery device (e.g., a catheter). Once deployed within the body at a target location, the implant 600 may assume the form shown in FIG. 6B to anchor the implant 600 to one or more tissue walls.

The structure and/or orientation of the central flow portion 602 may cause the first set 605a of anchoring arms 604 to be at least partially offset from the second set 605b of anchoring arms 604. For example, during delivery, the first anchoring arm 604a and/or the second anchoring arm 604b may be configured to extend linearly along a horizontal axis 630 (e.g., as shown in FIG. 6A). After removal from a delivery device (e.g., a catheter), the first anchoring arm 604a and/or the second anchoring arm 604b may be configured to extend generally perpendicularly to the horizontal axis 630 (e.g., as shown in FIG. 6B). The first set 605a of anchoring arms 604 and the second set 605b of anchoring arms 604 may be situated at different positions along the horizontal axis 630 due to the central flow portion 602 extending diagonally with respect to the horizontal axis 630 between the first set 605a and the second set 605b of anchoring arms 604.

The central flow portion 602 may be configured to at least partially bend to form an at least partially curved surface between sets 605 of anchoring arms 604. The curvature of the central flow portion 602 may advantageously increase the size of the central lumen of the implant 600 to allow for greater blood flow through the implant 600.

The natural expansion and/or bending of the anchoring arms 604 may advantageously increase an ease of use in delivery of the implant 600. For example, the implant 600 may be conformed as necessary (e.g., at least partially straightening the anchoring arms 604) for ease of use in delivering the implant via various sizes and/or types of delivery devices (e.g., different sizes of catheters). The implant 600 may be configured to be molded to any of a variety of forms while still allowing the implant 600 to expand to a pre-set form once delivered to a target location within a body of a patient.

### Delivery Processes

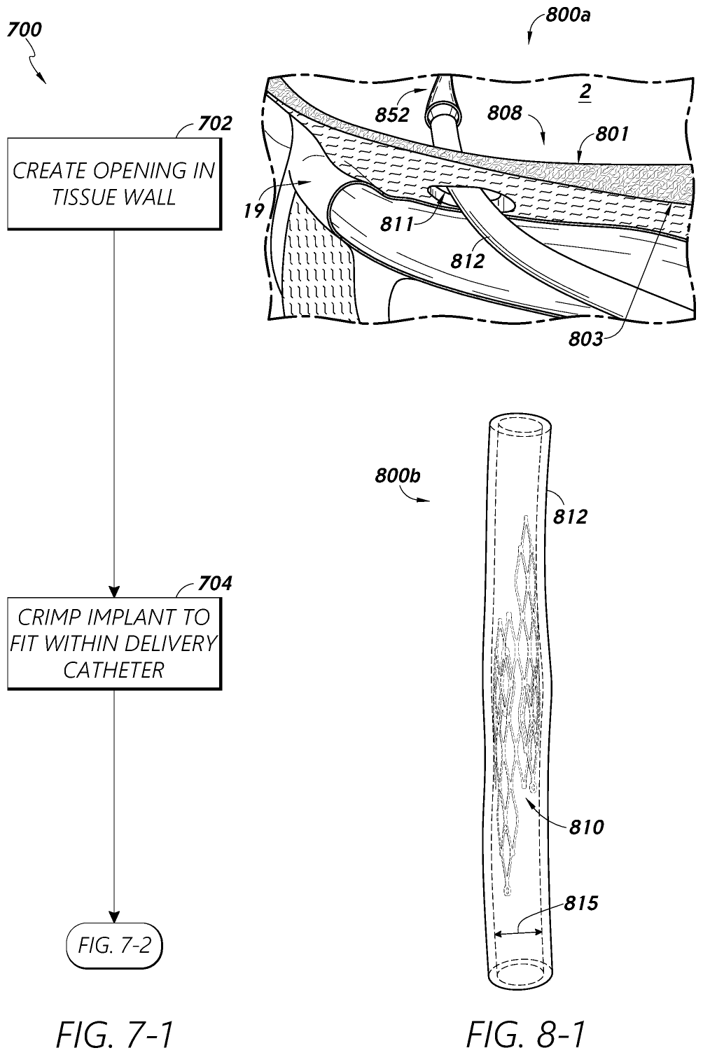

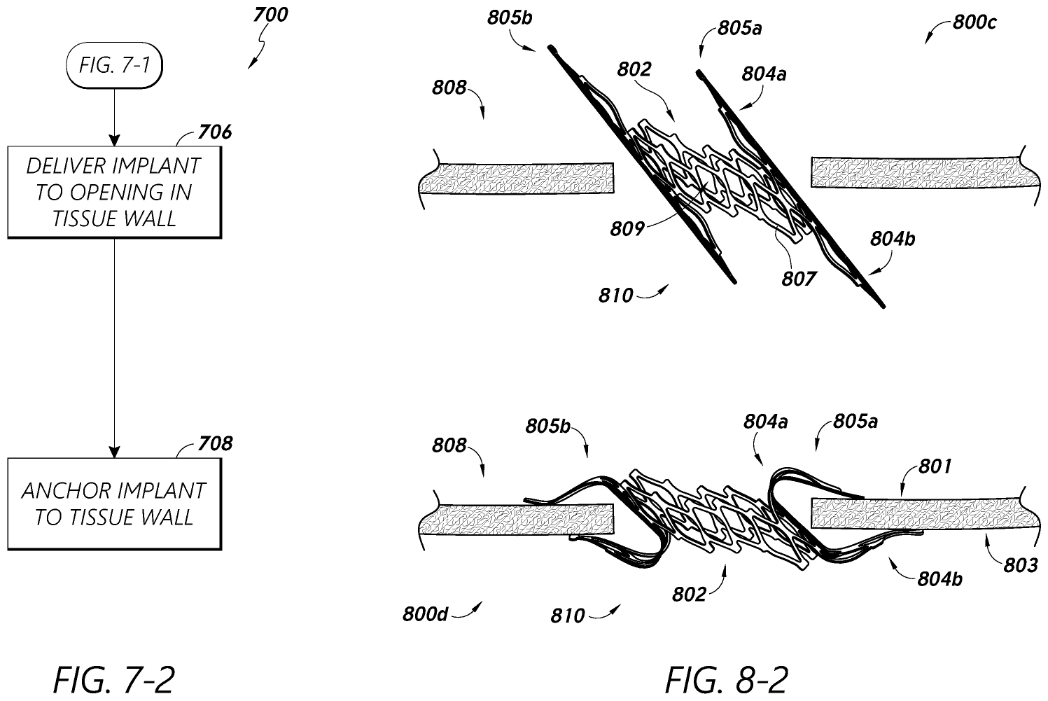

FIG. 7 (FIGS. 7-1 and 7-2) is a flow diagram illustrating a process for delivering and/or anchoring an implant to a treatment site in accordance with one or more embodiments of the present disclosure. FIG. 8 (FIGS. 8-1 and 8-2) provides several images associated with the process of FIG. 7 to illustrate aspects of the process according to one or more implementations thereof.

At block 702, the process involves creating an opening in a tissue wall. Image 800a of FIG. 8 is a side view of an opening (e.g., puncture hole) 811 through a tissue wall 808 (e.g., between the coronary sinus 19 and the left atrium 2) for placement of an implant in the opening 811. As shown in image 800a, an implant deployment or delivery catheter 812 may be advanced to the tissue wall 808 between two chambers (e.g., the coronary sinus 19 and the left atrium 2). The catheter 812 may have a soft and/or tapered distal tip 852. The delivery catheter 812 may be advanced through the opening 811 in the tissue wall 808 into, for example, the left atrium 2. The opening 811 may be created in any of a variety of ways. One example method is the following.

Initially, a guidewire may be advanced, for example, from the right atrium into the coronary sinus 19 through its ostium or opening. A catheter 812 may be advanced over the guidewire. The catheter 812 may be introduced into the body through a proximal end of an introducer sheath. An introducer sheath may provide access to the particular vascular pathway (e.g., jugular or subclavian vein) and may have a hemostatic valve therein. While holding the introducer sheath at a fixed location, the surgeon can manipulate the puncture catheter to the implant site. A puncture sheath having a puncture needle with a sharp tip may be advanced along a catheter 812 and punctured through the wall 808 into, for example, the left atrium 2. A puncture expander may be advanced along the guidewire and through the tissue wall 808 into the left atrium 2. The puncture expander may be, for example, an elongated inflatable balloon. The puncture expander may be inflated radially outward so as to widen the puncture through the tissue wall 808.

An implant may be delivered through a lumen of the catheter 812. During delivery, the implant may be in a collapsed configuration to facilitate delivery. For example, the implant may be bent, twisted, and/or otherwise configured to have a minimal profile to facilitate delivery through the catheter 812. The implant may be located in the annular space between an inner sheath and outer sheath of the catheter 812. An inner sheath may be retracted so that the implant is placed in intimate engagement with the tissue wall 808. Radiopaque markers may be provided to facilitate positioning of the catheter 812 and/or implant. By creating an opening 811 between the left atrium 2 and the coronary sinus 19, blood can flow from the left atrium 2 (which is usually >8 mmHg) to the coronary sinus 19 (which is usually <8 mmHg). One or more implants may be delivered and/or anchored to a first side 801 and/or to a second side 803 of the tissue wall 808.

At block 704, the process involves crimping the implant to fit the implant into a delivery catheter. The terms “catheter,” “delivery catheter,” and “sheath” are used herein according to their broad and ordinary meanings and may refer to any type of tube suitable for insertion in the body. “Catheter” and “sheath” may be used substantially interchangeably is some contexts herein. Image 800b of FIG. 8 illustrates an example implant 810 being delivered via a catheter 812, which may have a diameter 815 that is less than a diameter of the implant 810 prior to crimping of the implant 810. In some embodiments, the entire implant 810 may be placed into the catheter 812. However, only a portion of the implant 810 may be placed into the catheter 812 in some embodiments. While within the catheter 812, the catheter 812 may prevent expansion of the implant 810. For example, the implant 810 may be configured to naturally expand and/or naturally return to a pre-defined form that has a greater diameter/width than the diameter/width 815 of the catheter 812. However, the catheter 812 may be configured to prevent such expansion of the implant 810.

The implant 810 may be crimped prior to delivery into the body and/or prior to being placed into the catheter 812. In some embodiments, crimping may involve pressing the sides of the implant 810 inwards to reduce the width of the implant 810 until the width of the implant 810 is equal to or less than the width 815 of the inner lumen of the catheter 812. Additionally or alternatively, crimping may involve pulling the ends of the implant 810 to increase a length of the implant 810.

At block 706, the process involves delivering the implant to the created opening in the tissue wall. Image 800c illustrates an implant 810 following delivery to an opening in a tissue wall 808. Delivering the implant 810 may involve removing the implant 810 from the catheter 812 when the catheter 812 is delivered to the implant location. In some embodiments, removing the implant 810 may involve pulling back at least a portion of the catheter 812 to expose the implant 810 to the blood vessel and/or other portion of the body. When the implant 810 is exposed from the catheter 812, the implant 810 may at least partially expand and/or the anchoring arms 804 may at least partially bend. In some embodiments, the implant 810 may be expanded manually and/or may be configured to naturally expand upon being at least partially removed from the catheter 812. However, expansion of the implant 810 may be assisted at least in part. For example, the catheter 812 and/or another surgical tool may be used to press against and/or pull the implant 810 to move the implant 810 towards an expanded shape and/or position.

The implant 810 may include a central flow portion 802 configured to fit at least partially within the opening 811 in the tissue wall. The central flow portion 802 may be configured to extend along sides of the opening 811 to maintain the opening 811 and/or to prevent ingrowth of tissue. The central flow portion 802 may be configured to allow blood flow through the implant 810. In some embodiments, the anchoring arms 804 may be configured to continue extend from the central flow portion 802 and/or may be configured to maintain the opening 811. The central flow portion 802 and/or anchoring arms 804 may comprise a network and/or pattern of struts 807 configured to form two or more cells 809 allowing blood flow through the central flow portion 802 and/or anchoring arms 804.

While within a delivery device and/or immediately following removal from a delivery device, the implant 810 may be held in the straightened and/or stretched form shown in image 800c, in which the anchoring arms 804 (e.g., a first anchoring arm 804a and/or a second anchoring arm 804b) may be at least partially straightened such that the first anchoring arm 804a and the second anchoring arm 804b form a continuous line. The implant may be shape-set in an at least partially bent form such that the anchoring arms 804 may naturally bend when crimping force is removed.

At block 708, the process 700 involves anchoring the implant to the tissue wall. Image 800d of FIG. 8 illustrates an implant 810 anchored to a tissue wall 808 via anchoring arms 804 of the implant 810. In some embodiments, a first anchoring arm 804a may be configured to contact and/or attach to a first side 801 of the tissue wall 808 and/or a second anchoring arm 804b may be configured to contact and/or attach to a second side 803 of the tissue wall 808. The first anchoring arm 804a and the second anchoring arm 804b may be configured to establish a pincer grip on the tissue wall 808 by pressing simultaneously against the first side 801 of the tissue wall 808 and the second side 803 of the tissue wall 808, respectively.

The implant 810 may comprise two sets of anchoring arms 804, each configured to anchor to different portions of the tissue wall 808. For example, a first set 805a of anchoring arms 804 (including the first anchoring arm 804a and the second anchoring arm 804b) may be configured to establish a pincer grasp on a first portion of the tissue wall 808 while a second set 805b of anchoring arms may be configured to establish a pincer grasp on a second portion of the tissue wall 808.

In some embodiments, one or more anchoring arms 804 may be configured to penetrate and/or hook onto the tissue wall 808. For example, one or more anchoring arms 804 may comprise one or more hooks extending from the anchoring arms 804 configured to penetrate the tissue wall 808 to establish a more secure attachment to the tissue wall 808.

The one or more anchoring arms 804 may be configured to naturally bend towards the tissue wall 808 upon delivery at the opening 811 in the tissue wall 808. For example, when the implant 810 is removed from a delivery device (e.g., a catheter), the implant 810 may naturally assume the form shown in image 800d. In some embodiments, the implant 810 may be shape-set and/or may be at least partially composed of a shape-memory alloy (e.g., Nitinol). For example, the implant 810 may be shape-set such that the first anchoring arm 804a and the second anchoring arm 804b may be configured to be pressed together and/or near each other such that when the tissue wall 808 is situated between the first anchoring arm 804a and the second anchoring arm 804b, the first anchoring arm 804a and the second anchoring arm 804b may be configured to securely pinch the tissue wall 808.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A medical implant comprising:
a central flow portion having a first network of struts forming two or more cells;
a first set of anchoring arms; and
a second set of anchoring arms;
wherein the two or more cells are arranged linearly and extend from the first set of anchoring arms to the second set of anchoring arms.

2. The medical implant of claim 1, wherein each strut of the first network of struts is configured to bend in response to crimping pressure such that the two or more cells increase in length and decrease in width.

3. The medical implant of claim 2, wherein each strut of the first network of struts is configured to return to an original configuration in response to removal of the crimping pressure.

4. The medical implant of claim 3, wherein a diameter of the central flow portion is configured to reduce in response to the crimping pressure.

5. The medical implant of claim 4, wherein the first set of anchoring arms has a second network of struts forming cells.

6. The medical implant of claim 5, wherein the first set of anchoring arms includes a first anchoring arm and a second anchoring arm configured to anchor to a tissue wall using a pincer grasp.

7. The medical implant of claim 6, wherein the first anchoring arm and the second anchoring arm have an at least partially bent form.

8. The medical implant of claim 7, wherein the first anchoring arm and the second anchoring arm are configured to be at least partially straightened during delivery into a patient's body.

9. The medical implant of claim 8, wherein the central flow portion is further configured to prevent in-growth of tissue through the central flow portion.

10. The medical implant of claim 9, wherein the central flow portion is configured to expand in response to expansion of a tissue wall.

11. The medical implant of any of claims 1-10, wherein the central flow portion is configured to fit at least partially within an opening in a tissue wall, wherein: the tissue wall is situated between a first anatomical chamber and a second anatomical chamber and the opening represents a blood flow path between the first anatomical chamber to the second anatomical chamber, and to maintain the blood flow path from the first anatomical chamber to the second anatomical chamber.

12. A method comprising:
crimping a medical implant to reduce a profile of the medical implant, the medical implant comprising a network of struts forming two or more cells, wherein crimping the medical implant causes at least some struts of the network of struts to bend;
inserting the medical implant into a catheter;
delivering the catheter to a treatment location within a human body; and
removing the medical implant from the catheter.

13. The method of claim 12, wherein the medical implant further comprises a first set of anchoring arms and a second set of anchoring arms.

14. The method of claim 13, wherein the two or more cells are arranged linearly from the first set of anchoring arms to the second set of anchoring arms.

15. The method of claim 14, wherein removing the medical implant from the catheter causes expansion of the medical implant to an expanded profile and a width of the medical implant in the expanded profile exceeds a width of the catheter.

16. The method of claim 15, wherein the medical implant is at least partially composed of Nitinol.

17. A medical implant comprising:
a continuous network of struts forming:
a cylindrical central flow portion allowing blood flow through an interior lumen of the central flow portion;
a first set of anchoring arms extending from the central flow portion; and
a second set of anchoring arms extending from the central flow portion;
wherein the central flow portion comprises a first set of two or more cells extending linearly between a first side of the first set of anchoring arms and a first side of the second set of anchoring arms.

18. The medical implant of claim 17, wherein the central flow portion further comprises a second set of two or more cells extending linearly between a second side of the first set of anchoring arms and a second side of the second set of anchoring arms.

19. The medical implant of claim 18, wherein the first set of anchoring arms and the second set of anchoring arms are connected to each other only via the first set of two or more cells and the second set of two or more cells.

20. The medical implant of claim 19, wherein the first set of anchoring arms comprises a first anchoring arm and a second anchoring arm configured to bend at least partially to establish a pincer grasp at a tissue wall.
